(19) United States
(12) Patent Application Publication
(10) Pub. No.: US 2026/0041616 A1
(43) Pub. Date: Feb. 12, 2026

(54) Dental Care Solution and Manufacturing Method Thereof

(71) Applicants: National Taipei University of Technology, Taipei (TW); Taipei Medical University, Taipei (TW)

(72) Inventors: Ren-Jei CHUNG, Taipei City (TW); Yu-Chien LIN, Taipei City (TW); Wan-Rong LIN, Taipei City (TW); Nai-Chia TENG, Taipei City (TW); Jen-Chang YANG, Taipei City (TW)

(21) Appl. No.: 19/293,032

(22) Filed: Aug. 7, 2025

(30) Foreign Application Priority Data
Aug. 7, 2024 (TW) 113129528

(51) Int. Cl. A61K 8/19; A61K 8/21; A61K 8/27; A61Q 11/00

(52) U.S. Cl. CPC A61K 8/19; A61K 8/21; A61K 8/27; A61Q 11/00; A61K 2800/92

(57) ABSTRACT

The present invention is a dental care solution and manufacturing method thereof. By using silver nanoparticles and mixing zinc ions, titanium ions, or silicon ions to improve the dental care solution stability, the present invention has antibacterial effect and effectively assists preventing tooth decay.

### BACKGROUND OF THE INVENTION

### 1. Field of the Invention

The present invention relates to a solution and a method for manufacturing thereof, and more particularly to a dental care solution and a method for manufacturing thereof.

### 2. Description of the Prior Art

Common clinical methods for preventing the dental caries normally include the use of dental fillings and fluoride. For patients at high risk of dental caries and with deep grooves on the chewing surfaces of the teeth, the use of sealing agents is an effective minimally invasive treatment which is quite good cost-effective, usually does not require any local anesthesia, and the use of sealing agents can be used when natural tooth structure is removed.

In the past years, amalgam fillings were commonly used in clinical practice. There is excellent durability and strength for amalgam fillings, but there are amalgam fillings health concerns because the amalgam will continuously release low levels of mercury vapor. In recent years, composite resin fillings have been used, which can be subdivided into methyl methacrylate (MMA)-based resin fillings and glass ionomer-based fillings, which also can form a mechanical barrier between tooth enamel and pathogenic biofilm. And composite resin fillings are strong adhesion to the tooth surface, so that composite resin fillings can protect the teeth. For people at low risk of tooth decay, over-the-counter fluoride toothpaste is a good daily oral care solution.

In addition, due to the tiny size of silver nanoparticles, the amount of silver ions released can be changed, and the surface energy can be interfered, and can be induced the generation of reactive oxygen species (ROS), so that the tiny size of silver nanoparticles further can affect the metabolism of bacteria, inhibits the activity of cellular proteins, and ultimately causes apoptosis of microorganisms. Therefore, the silver nanoparticles can be regarded as a powerful antibacterial agent and can be widely used in the field of dental restorative materials and dentures.

### SUMMARY OF THE INVENTION

The present invention is a method for forming a dental care solution, which uses silver nanoparticles, mixed with zinc ions, titanium ions, or silicon ions to improve the stability of the tooth care solution, and has an antibacterial effect, which can effectively assist in preventing tooth decay.

The present invention provides a method for forming a dental care solution, which is to mix silver nanoparticles with a solution containing silver, zinc, fluoride ion solution or chloride ion solution.

The present invention provides a method for forming a dental care solution, which comprises mixing silver nanoparticles with a solution containing silver, titanium, fluoride ion solution or chloride ion solution.

The present invention provides a method for forming a dental care solution, which comprises mixing silver nanoparticles with a solution containing silver, silicon, fluoride ion solution or chloride ion solution.

The present invention provides a method for forming a dental care solution, which comprises mixing silver nanoparticles with zinc, a fluoride ion solution or a chloride ion solution.

The present invention provides a method for forming a dental care solution, which comprises silver nanoparticles with titanium, a fluoride ion solution or a chloride ion solution.

The present invention provides a method for forming a dental care solution, which comprises mixing silver nanoparticles with silicon, a fluoride ion solution or a chloride ion solution.

The present invention is a method for forming a dental care solution, which comprises retaining the use of fluoride ions or chloride ions in an appropriate proportion, and further comprises silver ions, as well as the preparation of titanium ion solution, zinc ion solution, and silicon ion solution, also uses a hydrothermal method to synthesize silver nanoparticles, and, and the silver nanoparticles are synthesized by hydrothermal method and then used after adding in appropriate concentration.

One of the advantages of the method for forming a dental care solution of the present invention is that the particle size of the silver nanoparticles is smaller than the diameter of the human dentinal tubules, so that the silver nanoparticles can directly entre into the tubules to release silver ions, achieve a long-term antibacterial effect.

One of the advantages of the method of forming a dental care solution of the present invention is that there is a significant antibacterial effect against Staphylococcus aureus (S. aureus), Escherichia coli (E. coli), Porphyromonas gingivalis (P. gingivalis), and Enterococcus faecalis (E. faecalis).

One of the advantages of the method for forming a dental care solution of the present invention is that when the tooth slices after coating are soaked in artificial saliva for a cycle, metal elements can still be detected to remain in the tooth substance, proving that the invention can be combined with the teeth to achieve effective functions.

One of the advantages of the method for forming a dental care solution of the present invention is that the tooth staining at the carious part after application is much milder than that of the conventional method, which can increase the willingness of patients to choose.

### BRIEF DESCRIPTION OF THE DRAWINGS

The foregoing aspects and advantages of this invention will become more apparent from the following detailed description, in conjunction with the accompanying drawings, wherein:

FIG. 1 schematically shows a method for synthesizing silver nanoparticles according to the present invention.

FIG. 2A schematically shows a method for forming a solution containing silver, zinc, and fluoride ion solution or chloride ions according to the present invention.

FIG. 2B schematically shows a method for forming a solution containing zinc, and fluoride ion solution or chloride ions according to the present invention.

FIG. 3A schematically shows a method for forming a solution containing silver, titanium, and fluoride ion solution or chloride ions according to the present invention.

FIG. 3B schematically shows a method for forming a solution containing titanium, and fluoride ion solution or chloride ions according to the present invention.

FIG. 4A schematically shows a method for forming a solution containing silver, silicon, and fluoride ion solution or chloride ions according to the present invention.

FIG. 4B schematically shows a method for forming a solution containing silicon, and fluoride ion solution or chloride ions according to the present invention.

FIG. 5 is an X-ray diffractometer analysis diagram of a method for forming a dental care solution according to the present invention.

FIG. 6 is a field emission scanning electron microscope image of the morphology of silver nanoparticles formed by a method for forming a dental care solution according to the present invention.

### DESCRIPTION OF THE PREFERRED EMBODIMENT

The method of forming a dental care solution of the present invention comprises the following steps: firstly, adding 0.5 mg to 1.5 mg of silver nanoparticles, mixing with 5 ml to 15 ml silver, zinc, and fluoride ion solution or chloride ion solution, then taking out the silver nanoparticles with a concentration of 0.05 mg to 0.15 mg, and the 5 ml to 15 ml silver, zinc, and fluoride ion solution or chloride ion solution.

The method of forming a dental care solution of the present invention comprises the following steps: firstly, adding 0.5 mg to 1.5 mg of silver nanoparticles, mixing with 5 ml to 15 ml of zinc, and fluoride ion solution or chloride ion solution, then taking out the silver nanoparticles with a concentration of 0.05 mg to 0.15 mg, and the 5 ml to 15 ml of zinc and fluoride ion solution or chloride ion solution.

The present invention provides a method for forming a dental care solution, comprising the following steps: firstly, adding 0.5 mg to 1.5 mg of silver nanoparticles, mixing with 5 ml to 15 ml silver, titanium, and fluoride ion solution or chloride ion solution, and then taking out the silver nanoparticles with a concentration of 0.05 mg to 0.15 mg, and the 5 ml to 15 ml silver, titanium, and fluoride ion solution or chloride ion solution.

The present invention provides a method for forming a dental care solution, comprising the following steps: firstly, adding 0.5 mg to 1.5 mg of silver nanoparticles, mixing with 5 ml to 15 ml of titanium, and fluoride ion solution or chloride ion solution, and then taking out the silver nanoparticles with a concentration of 0.05 mg to 0.15 mg, and the 5 ml to 15 ml of titanium, and fluoride ion solution or chloride ion solution.

The present invention provides a method for forming a tooth care solution, comprising the following steps: firstly, adding 0.5 mg to 1.5 mg of silver nanoparticles, mixing with 5 ml to 15 ml silver, silicon, and fluoride ions or chloride ions, then taking out the silver nanoparticles with a concentration of 0.05 mg to 0.15 mg and the 5 ml to 15 ml silver, silicon, and fluoride ions or chloride ions.

The present invention provides a method for forming a tooth care solution, comprising the following steps: firstly, adding 0.5 mg to 1.5 mg of silver nanoparticles, mixing with 5 ml to 15 ml silicon and fluoride ion solution or chloride ion solution, then taking out the silver nanoparticles with a concentration of 0.05 mg to 0.15 mg and the 5 ml to 15 ml of silicon, and fluoride ion solution or chloride ion solution, and mixing the mixture with the mixture.

A method for synthesizing silver nanoparticles

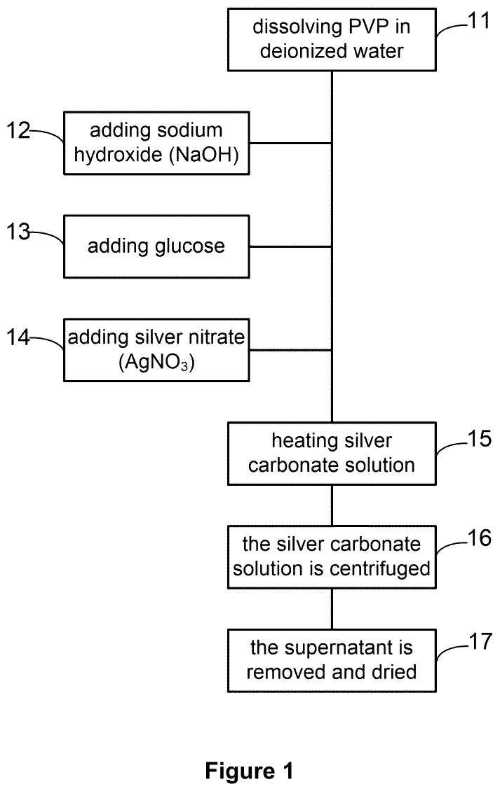

FIG. 1 shows a method for synthesizing silver nanoparticles. As shown in step 11 of FIG. 1, firstly, 0.3 g to 0.7 g of polyvinylpyrrolidone (PVP) is dissolved in 5 ml to 15 ml of deionized water (DI Water) and is stirred for 25 minutes to 35 minutes.

As shown in step 12 of FIG. 1, 3 ml to 7 ml of 0.1 M sodium hydroxide (NaOH) is added and is stirred for 5 minutes to 15 minutes.

As shown in step 13 of FIG. 1, 3 ml to 7 ml of 0.2 M glucose is added and is stirred for 5 minutes to 15 minutes.

As shown in step 14 of FIG. 1, 5 ml to 15 ml of 0.05 M silver nitrate (AgNO₃) is added continuously and stirred for 25 to 35 minutes to form a silver carbonate (Ag₂CO₃) solution.

As shown in step 15 of FIG. 1, the silver carbonate solution is then heated using a hydrothermal method, the temperature is maintained at 120° C., and the temperature is maintained for 25 minutes to 35 minutes, 55 minutes to 65 minutes, 110 minutes to 130 minutes, and 230 minutes to 250 minutes.

As shown in step 16 of FIG. 1, after the silver carbonate solution is cooled, the silver carbonate solution is centrifuged for 5 minutes to 15 minutes at a speed of 8500 per minute to 9500 per minute. The centrifugation washing needs to be performed at 2 times to 5 times.

As shown in step 17 of FIG. 1, the supernatant is removed, and the mixture is placed in an oven at 45° C. to 55° C. for drying to form silver nanoparticles.

Therefore, the chemical formula synthesized is as follows:

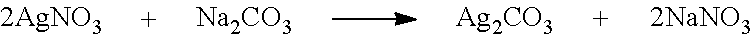

A method for preparing and manufacturing a solution containing silver, zinc, and fluoride ion solution or chloride ion solution:

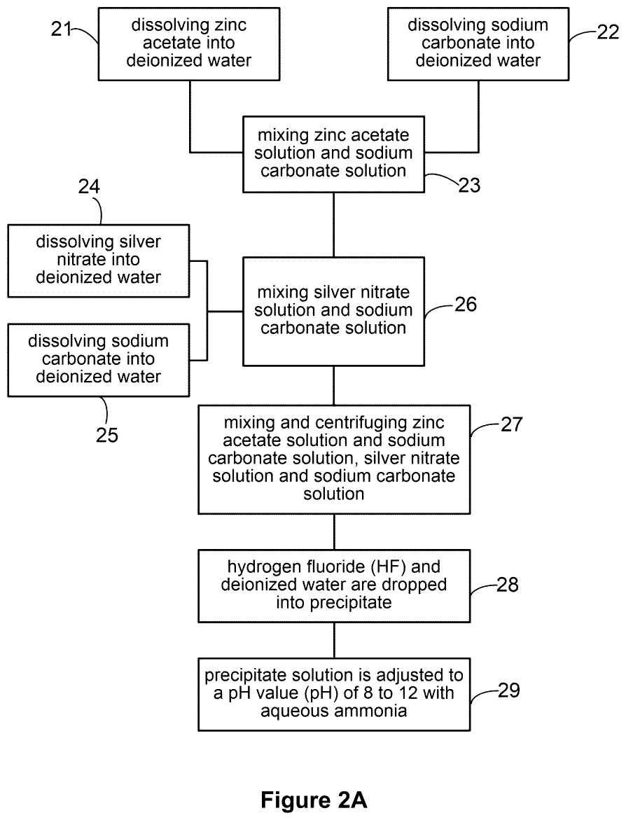

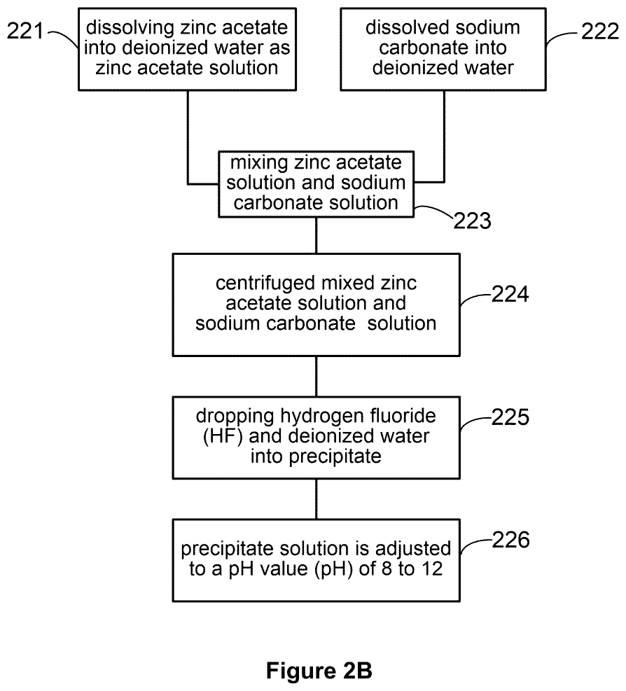

FIG. 2A shows a method for preparing and manufacturing a solution containing silver, zinc, and fluoride ion solution or chloride ion solution. As shown in step 21 of FIG. 2A, 1.695 g to 2.695 g of zinc acetate is dissolved into 3 ml to 7 ml of deionized water to form a zinc acetate solution.

As shown in step 22 of FIG. 2A, 2.07 g to 2.17 g of sodium carbonate is dissolved into 5 ml to 15 ml of deionized water to form a sodium carbonate solution.

As shown in step 23 of FIG. 2A, the zinc acetate solution and the sodium carbonate solution are mixed and stirred for 25 minutes to 35 minutes.

As shown in step 24 of FIG. 2A, 1.02 g to 1.12 g of silver nitrate is dissolved into 2 ml to 5 ml of deionized water to form a silver nitrate solution.

As shown in step 25 of FIG. 2A, 0.63 g to 0.73 g of sodium carbonate is dissolved into 5 ml to 15 ml of deionized water to form a sodium carbonate solution.

As shown in step 26 of FIG. 2A, the silver nitrate solution and the sodium carbonate solution are mixed and stirred for 25 minutes to 35 minutes.

As shown in step 27 of FIG. 2A, the zinc acetate solution and the sodium carbonate solution, as well as the silver nitrate solution and the sodium carbonate solution are mixed and centrifuged for 5 minutes to 15 minutes at a speed of 8500 per minute to 9500 per minute. The centrifugation is performed at 2 times to 5 times to remove the supernatant and leave the precipitate.

As shown in step 28 of FIG. 2A, 2 ml to 4 ml of hydrogen fluoride (HF) and 2 ml to 4 ml of deionized water are dropped into the precipitate to form a precipitate solution.

As shown in step 29 of FIG. 2A, the precipitate solution is adjusted to a pH value (pH) of 8 to 12 with aqueous ammonia.

As mentioned above in step 28 of FIG. 2A, the hydrogen fluoride may also be used as an alternative to chlorine fluoride to generate chloride ions.

Therefore, the synthesized chemical formula is as follows:

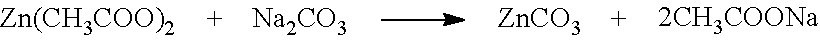

A solution containing silver, zinc, fluoride ion solution or chloride ion solution according to the aforementioned method for preparing and manufacturing a silver, zinc, and fluoride ion solution or a chloride ion solution, wherein the concentration of the solution is as follows: the silver ion concentration ranges from 10,000 parts per million (ppm) to 100,000 ppm, and the zinc ion concentration ranges from 1,000 ppm to 10,000 ppm.

The present invention discloses a solution containing silver, zinc, fluoride ion solution or chloride ion solution, which is a solution with caring properties. A lower silver ion concentration is used to improve tooth staining after the treatment. The introduction of zinc ions can enhance the antibacterial effect of the solution and provide protein composition. In addition, there are the antibacterial properties, fluoride ions can also help re-mineralize carious tooth tissue.

A method for preparing and manufacturing a solution containing zinc, and fluoride ion solution or chloride ion solution:

FIG. 2B shows a method for preparing and manufacturing a solution containing zinc, fluoride ions or chloride ions. As shown in step 221 of FIG. 2B, 1.695 g to 2.695 g of zinc acetate is dissolved into 3 ml to 7 ml of deionized water to form as a zinc acetate solution.

As shown in step 222 of FIG. 2B, 2.07 g to 2.17 g of sodium carbonate is dissolved into 5 ml to 15 ml of deionized water to form as a sodium carbonate solution.

As shown in step 223 of FIG. 2B, the zinc acetate solution and the sodium carbonate solution are mixed and stirred for 25 minutes to 35 minutes.

As shown in step 224 of FIG. 2B, the mixed zinc acetate solution and the sodium carbonate solution are centrifuged for 5 minutes to 15 minutes at a speed of 8500 revolutions per minute to 9500 revolutions per minute. The centrifugation is performed at 2 times to 5 times, to remove the supernatant and leave the precipitate.

As shown in step 225 of FIG. 2B, 2 ml to 4 ml of hydrogen fluoride (HF) and 2 ml to 4 ml of deionized water are dropped into the precipitate to form as a precipitate solution.

As shown in step 226 of FIG. 2B, the precipitate solution is adjusted to a pH value (pH) of 8 to 12 with aqueous ammonia.

As described above in step 225 of FIG. 2B, the hydrogen fluoride may also be used as chlorine fluoride to generate chloride ions.

A solution containing zinc, fluoride ion solution or chloride ion solution according to the abovementioned method for preparing and manufacturing a zinc, fluoride ion solution or chloride ion solution, wherein the concentration of the solution is as follows: the zinc ion concentration ranges from 1,000 parts per million (ppm) to 10,000 ppm.

A solution containing zinc, fluoride ion solution or chloride ion solution of the present invention is a solution with the caring property. Wherein, the antibacterial properties of the contained zinc ions, fluoride ions or chloride ions can be performed. The zinc ions also provide the composition of tooth proteins. The fluoride ions can further help to re-mineralize the carious tooth substance.

A method for preparing and manufacturing a solution containing silver, titanium, and fluoride ion solution or chloride ion solution:

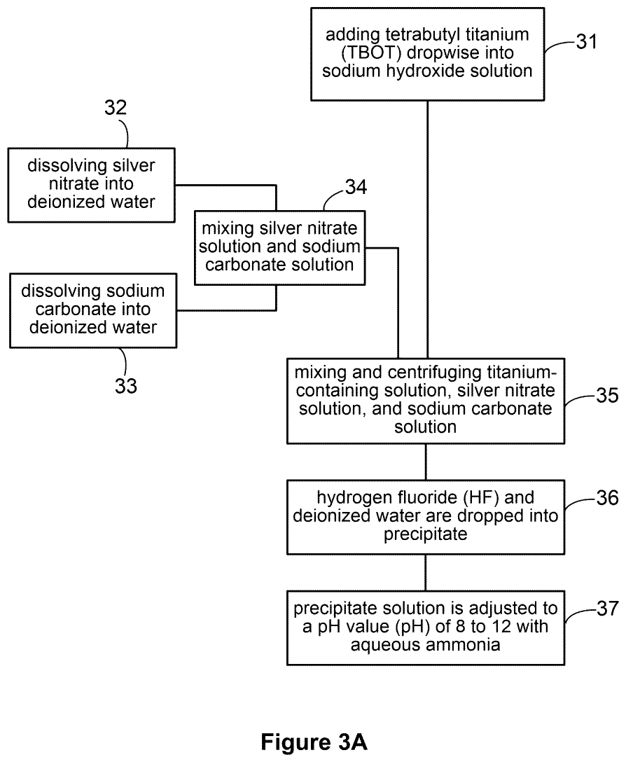

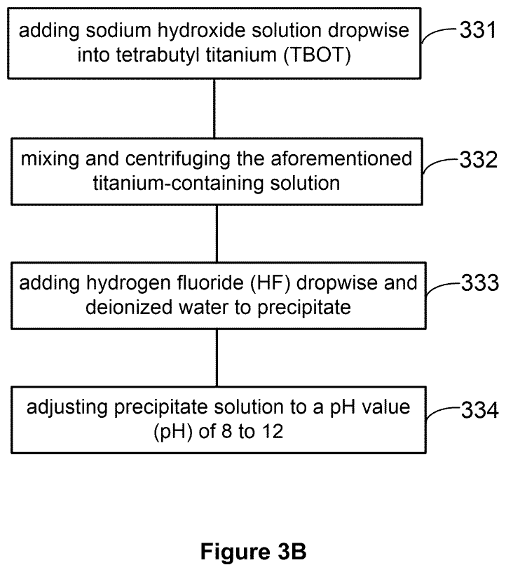

FIG. 3A shows a method for preparing and manufacturing a solution containing silver, titanium, and fluoride ion solution or chloride ion solution. As shown in step 31 of FIG. 3A, 1 ml of tetrabutyl titanium (TBOT) is added dropwise into 10 ml to 20 ml of 1M sodium hydroxide solution to form a solution containing titanium and is stirred for 55 to 65 minutes.

As shown in step 32 of FIG. 3A, 1.02 g to 1.12 g of silver nitrate is dissolved into 1 ml to 5 ml of deionized water to form as a silver nitrate solution.

As shown in step 33 of FIG. 3A, 0.63 g to 0.73 g of sodium carbonate is dissolved into 5 ml to 15 ml of deionized water to form as a sodium carbonate solution.

As shown in step 34 of FIG. 3A, the silver nitrate solution and the sodium carbonate solution are mixed and stirred for 25 minutes to 35 minutes.

As shown in step 35 of FIG. 3A, the abovementioned titanium-containing solution, the silver nitrate solution, and the sodium carbonate solution are mixed and centrifuged for 5 minutes to 15 minutes at a speed of 8500 revolutions per minute to 9500 revolutions per minute. The centrifugation needs to be performed at 2 times to 5 times, and the supernatant is removed to leave the precipitate.

As shown in step 36 of FIG. 3A, 2 ml to 4 ml of hydrogen fluoride (HF) and 2 ml to 4 ml of deionized water are added dropwise to the precipitate to form a precipitate solution.

As shown in step 37 of FIG. 3A, the precipitate solution is adjusted to a pH value (pH) of 8 to 12 with aqueous ammonia.

Therefore, the synthesized chemical formula is as follows:

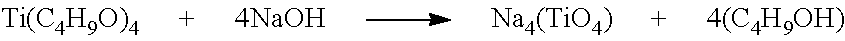

As in step 36 of FIG. 3A above, the hydrogen fluoride may alternatively be used as chlorine fluoride to generate chloride ions.

A solution containing silver, titanium, fluoride ion solution or chloride ion solution according to the aforementioned method for preparing and manufacturing a silver, titanium, fluoride ion solution or chloride ion solution, wherein the concentration of the solution is as follows: a silver ion concentration range of 10,000 parts per million (ppm) to 100,000 ppm, a titanium ion concentration range of 100 ppm to 1,000 ppm.

A solution containing silver, titanium, fluoride ion solution or chloride ion solution of the present invention is a solution with caring properties. A lower silver ion concentration is used to improve the staining condition after implementation. The purpose of using titanium ions is to make the solution resistant to discoloration and to assist in occluding the dentinal tubules exposed by caries. In addition, having antibacterial properties, fluoride ions can also help re-mineralize carious tooth tissue.

A method for preparing and manufacturing a solution containing titanium, and fluoride ion solution or chloride ion solution:

FIG. 3B shows a method for preparing and manufacturing a solution containing titanium, and fluoride ion solution or chloride ion solution. As shown in step 331 of FIG. 3B, 10 ml to 20 ml of 1M sodium hydroxide solution is added dropwise to 1 ml of tetrabutyl titanium (TBOT) to form the solution containing titanium, and the mixture is stirred for 55 minutes to 65 minutes.

As shown in step 332 of FIG. 3B, the aforementioned titanium-containing solution is mixed and centrifuged at 8500 rpm to 9500 rpm for 5 minutes to 15 minutes. Centrifugation is performed at 2 times to 5 times, and the supernatant is removed to leave the precipitate.

As shown in step 333 of FIG. 3B, 2 ml to 4 ml of hydrogen fluoride (HF) and 2 ml to 4 ml of deionized water are added dropwise to the precipitate to form a precipitate solution.

As shown in step 334 of FIG. 3B, the precipitate solution is adjusted to a pH value (pH) of 8 to 12 with aqueous ammonia.

As in step 333 of FIG. 3B, the hydrogen fluoride may alternatively be used as chlorine fluoride to generate chloride ions.

A solution containing titanium, and fluoride ion solution or chloride ion solution according to the abovementioned preparing and manufacturing a solution containing titanium, and fluoride ion solution or chloride ion solution, wherein the concentration of the solution is as follows: the titanium ion concentration ranges from 100 parts per million (ppm) to 1,000 parts per million (ppm).

A solution containing titanium, and fluoride ion solution or chloride ion solution of the present invention is a solution with a caring property, wherein the fluoride ion or chloride ion has an antibacterial property, and the titanium ion provides the solution with anti-discoloration property and assists in occluding the dentin tubules exposed by caries, and the fluoride ion can further assist in the remineralization of carious tooth substance.

A method for preparing and manufacturing a solution containing silver, silicon, and fluoride ion solution or chloride ion solution:

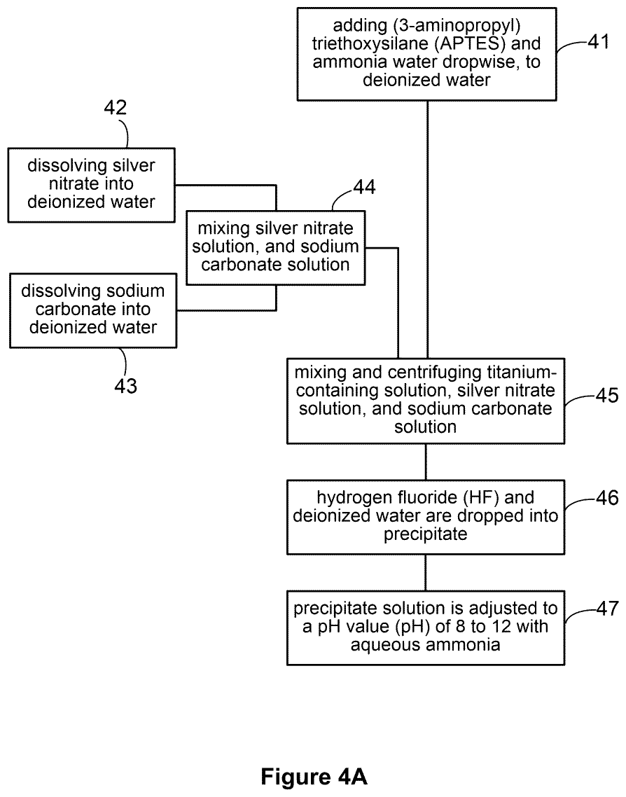

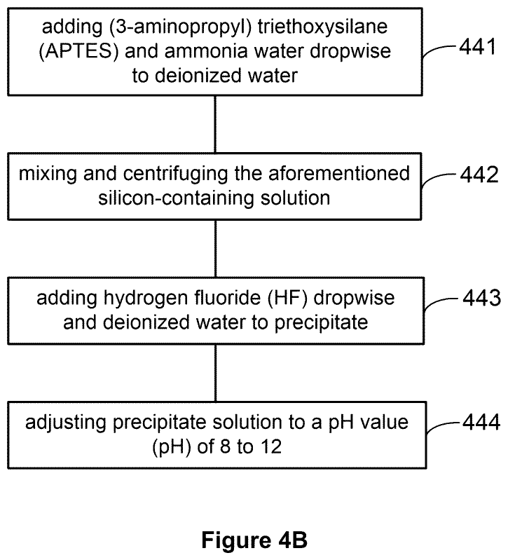

FIG. 4A shows a method for preparing and manufacturing a solution containing silver, silicon, and fluoride ion solution or chloride ion solution. As shown in step 41 of FIG. 4, 0.49 ml to 0.59 ml of (3-aminopropyl) triethoxysilane (APTES) and 0.5 ml to 1.5 ml of ammonia water are added dropwise to 2 ml to 6 ml of deionized water and stirred for 55 minutes to 65 minutes to form a silicon-containing solution.

As shown in step 42 of FIG. 4A, 1.02 g to 1.12 g of silver nitrate is dissolved into 1 ml to 5 ml of deionized water to form a silver nitrate solution.

As shown in step 43 of FIG. 4A, 0.63 g to 0.73 g of sodium carbonate is dissolved into 5 ml to 15 ml of deionized water to form a sodium carbonate solution.

As shown in step 44 of FIG. 4A, the silver nitrate solution and the sodium carbonate solution are mixed and stirred for 25 minutes to 35 minutes.

As shown in step 45 of FIG. 4A, the silicon silver nitrate solution, and the sodium carbonate solution are mixed and centrifuged at 8500 rpm to 9500 rpm for 5 minutes to 15 minutes, centrifuge and wash at 2 times to 5 times, the supernatant is removed, and the sediment is leaved up.

As shown in step 46 of FIG. 4A, 2 ml to 4 ml of hydrogen fluoride (HF) and 2 ml to 4 ml of deionized water are added dropwise to the precipitate to form a precipitate solution.

As shown in step 47 of FIG. 4A, the precipitate solution is adjusted to a pH value (pH) of 8 to 12 with aqueous ammonia.

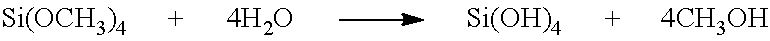

As in step 46 of FIG. 4B above, the hydrogen fluoride may alternatively be used as chlorine fluoride to generate chloride ions.

A solution containing silver, silicon, and fluoride ion solution or chloride ion solution according to the abovementioned method for preparing and manufacturing a solution containing silver, silicon, and fluoride ion solution or chloride ion solution, wherein the concentration of the solution is as follows: the titanium ion concentration ranges from 100 parts per million (ppm) to 1,000 parts per million (ppm).

The present invention is a solution containing silver, silicon, and fluoride ion solution or chloride ion solution, which is a solution with a caring property. The present invention mainly uses the lower silver ion concentration to improve the staining after implementation. In addition, silicon ions or fluoride ions have antibacterial properties and can help re-mineralize carious tooth substances.

A method for preparing and manufacturing a solution containing silicon, and fluoride ion solution or chloride ion solution:

FIG. 4B shows a method for preparing a silicon-containing fluoride ion solution or a chloride ion solution. As shown in step 441 of FIG. 4, 0.49 ml to 0.59 ml of (3-aminopropyl)triethoxysilane (APTES) and 0.5 ml to 1.5 ml of ammonia water are added dropwise to 2 ml to 6 ml of deionized water and stirred for 55 minutes to 65 minutes to form as a silicon solution.

As shown in step 442 of FIG. 4B, the mentioned silicon-containing solution is mixed and centrifuged at 8500 rpm to 9500 rpm for 5 minutes to 15 minutes. Centrifugation is performed at 2 times to 5 times, and the supernatant is removed to leave the precipitate.

As shown in step 443 of FIG. 4B, 2 ml to 4 ml of hydrogen fluoride (HF) and 2 ml to 4 ml of deionized water are added dropwise to the precipitate to form a precipitate solution.

As shown in step 444 of FIG. 4B, the precipitate solution is adjusted to a pH value (pH) of 8 to 12 with aqueous ammonia.

As shown in step 443 of FIG. 4B above, the hydrogen fluoride may also be used as an alternative to chlorine fluoride to generate chloride ions.

A solution containing silicon, fluoride ion solution or chloride ion solution according to the abovementioned method for preparing and manufacturing a solution containing silver, fluoride ion solution or chloride ion solution, wherein the concentration of the solution is as follows: the silicon ion concentration ranges from 1,000 parts per million (ppm) to 10,000 parts per million (ppm).

The present invention provides a solution containing silicon, and fluoride ion solution or chloride ion solution, which is a solution with a caring property, wherein the fluoride ions or chloride ions have antibacterial properties, and the silicon ions and fluoride ions can synergistically help the remineralization of carious tooth substances.

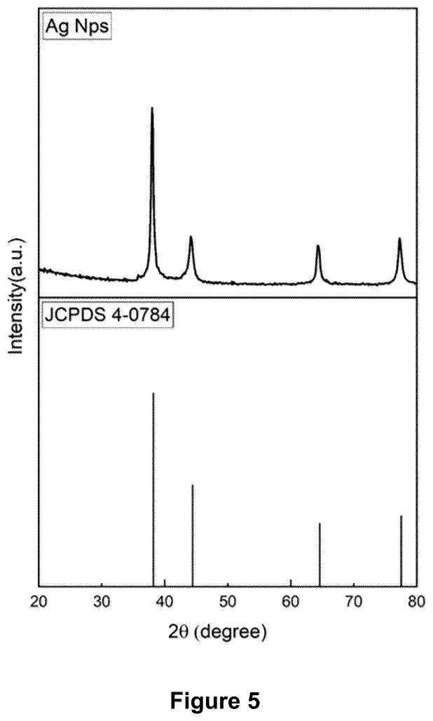

FIG. 5 is an X-ray diffractometer analysis diagram of a product formed by a method for forming a dental care solution according to the present invention. When the X-ray diffractometer is used for identification and analysis, the composition thereof contains silver nanoparticles (Ag). By comparing the composition with the standard spectrum of Ag in the JCPDs database (JCPDs cards 4-0784), so that the composition of the product is obtained by the method of the present invention contains silver nanoparticles.

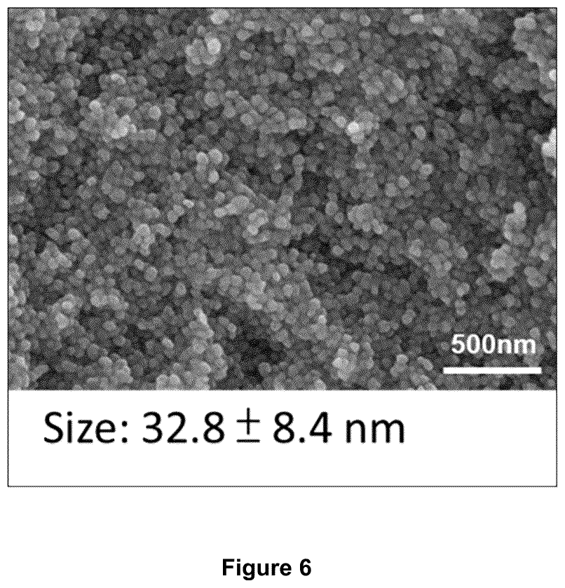

FIG. 6 is a field-emission scanning electron microscope (FE-SEM) image of a product formed by a method for forming a tooth care solution according to the present invention. When the field emission scanning electron microscope is used to scan and present an image, which is the surface of silver particles. Therefore, the product obtained is by the method of the present invention has silver particles.

In addition, the clinical use of the present invention is to first place cotton or gauze near the tooth with caries to prevent moisture from wetting the tooth, and, use a vacuum suction tool to remove moisture from the tooth surface, and then use a small brush to pick up an appropriate amount of the drug and apply it to the tooth cavity.

It will be appreciated that various modifications and adaptations may be made by those skilled-in-the-art without departing from the scope and spirit of this invention. Accordingly, the scope of the appended claims is not limited to the specific embodiments describe herein but, shall be construed to encompass all the features of patentable novelty inherent in the present invention, including all equivalents that would be recognized by those skilled in the art to witch this invention pertains.

What is claimed is:

1. A method of forming a dental care solution, comprising:
adding 0.5 mg to 1.5 mg of silver nanoparticles;
mixing 5 ml to 15 ml of a plurality of metal ions, and fluoride ion solution; and
taking out said silver nanoparticles with a concentration of 0.05 mg to 0.15 mg and said 5 ml to 15 ml solution containing said plurality of metal ions, and said fluoride ion solution.

2. The method according to claim 1, wherein said fluoride ion solution further comprises chloride ion solution.

3. The method according to claim 1, wherein said plurality of metal ions are selected from the group consisting of a silver ion and a zinc ion, a zinc ion, a silver ion and a titanium ion, a titanium ion, a silver ion and a silicon ion, and a silicon ion.

4. A method for synthesizing silver nanoparticles, comprising:
dissolving 0.3 g to 0.7 g of polyvinylpyrrolidone (PVP) in 5 ml to 15 ml of deionized water and stirring for 25 minutes to 35 minutes;
adding 3 ml to 7 ml of 0.1 M sodium hydroxide and stirring for 5 minutes to 15 minutes;
adding 3 ml to 7 ml of 0.2 M glucose and stirring for 5 minutes to 15 minutes;
adding 5 ml to 15 ml of 0.05 M silver nitrate and stirring for 25 minutes to 35 minutes to form a silver carbonate solution;
said silver carbonate solution being heated by a hydrothermal method, a temperature being maintained at 120° C., and said temperature being maintained for 25 minutes to 35 minutes, 55 minutes to 65 minutes, 110 minutes to 130 minutes, and 230 minutes to 250 minutes;
after said silver carbonate solution being cooled, centrifugation being performed for 5 minutes to 15 minutes at a speed of 8500 per minute to 9500 per minute, centrifugation is performed 2 times to 5 times; and
a supernatant being removed and a mixture being placed in an oven at 45° C. to 55° C. to be dried to obtain silver nanoparticles.

5. A method for preparing and manufacturing a solution containing a plurality of metal ions, and fluoride ions, comprising:
dissolving 1.695 g to 2.695 g of zinc acetate in 3 ml to 7 ml of deionized water to prepare a zinc acetate solution;
dissolving 2.07 g to 2.17 g of sodium carbonate in 5 ml to 15 ml of deionized water to prepare a sodium carbonate solution;
mixing said zinc acetate solution and said sodium carbonate solution, and stirring for 25 minutes to 35 minutes;
mixing a silver nitrate solution and a sodium carbonate solution, and stirring for 25 minutes to 35 minutes;
performing a centrifugal wash at 8500 revolutions per minute to 9500 revolutions per minute for 5 minutes to 15 minutes, and perform said centrifugal wash 2 times to 5 times to remove a supernatant and to leave a precipitate;
adding 2 ml to 4 ml of hydrogen fluoride or hydrogen chloride and 2 ml to 4 ml of deionized water dropwise to said precipitate to form a precipitate solution; and
adjusting said precipitate solution to a pH of 8 to 12 with ammonia water.

6. The method according to claim 5, wherein said plurality of metal ions comprises zinc ions.

7. The method according to claim 5, wherein said fluoride ion solution further comprises chloride ion solution.

8. The method according to claim 5, when said plurality of metal ions comprises silver ions and zinc ions, wherein, after a step for said mixing said zinc acetate solution and said sodium carbonate solution, and stirring for 25 minutes to 35 minutes, adding following steps as:
dissolving 1.02 g to 1.12 g of silver nitrate in 2 ml to 5 ml of deionized water to prepare a silver nitrate solution; and
dissolving 0.63 g to 0.73 g of sodium carbonate in 5 ml to 15 ml of deionized water to prepare a sodium carbonate solution”.

9. The method according to claim 5, wherein said silver ions comprise a silver ion concentration range, which is 10,000 parts per million (ppm) to 100,000 ppm, and zinc ions comprise a zinc ion concentration range, which is 1,000 ppm to 10,000 ppm.

10. A method for preparing and manufacturing a solution containing a plurality of metal ions, and fluoride ions, comprising:
adding 1 ml of tetrabutyltitanium dropwise into 10 ml to 20 ml of 1M sodium hydroxide solution to form a titanium-containing solution, and stirring for 55 minutes to 65 minutes;
dissolving 1.02 g to 1.12 g of silver nitrate in 1 ml to 5 ml of deionized water to prepare a silver nitrate solution;
dissolving 0.63 g to 0.73 g of sodium carbonate in 5 ml to 15 ml of deionized water to prepare a sodium carbonate solution;
mixing said silver nitrate solution and said sodium carbonate solution, and stirring for 25 minutes to 35 minutes;
performing a centrifugal wash at 8500 revolutions per minute to 9500 revolutions per minute for 5 minutes to 15 minutes, perform a centrifugal wash 2 times to 5 times, removing a supernatant, and leaving a precipitate;
adding 2 ml to 4 ml of hydrogen fluoride or hydrogen chloride and 2 ml to 4 ml of deionized water dropwise to said precipitate to form a precipitate solution; and
a precipitate solution is adjusted to a pH value of 8 to 12 with aqueous ammonia.

11. The method according to claim 10, wherein said plurality of metal ions comprises silver ions.

12. The method according to claim 10, wherein said fluoride ion solution further comprises chloride ion solution.

13. The method according to claim 10, when said plurality of metal ions comprises silver ions and titanium ions, wherein, after a step for adding 1 ml of tetrabutyltitanium dropwise into 10 ml to 20 ml of 1M sodium hydroxide solution to form a titanium-containing solution, and stirring for 55 minutes to 65 minutes, adding following steps as:
dissolving 1.02 g to 1.12 g of silver nitrate in 1 ml to 5 ml of deionized water to prepare a silver nitrate solution;
dissolving 0.63 g to 0.73 g of sodium carbonate in 5 ml to 15 ml of deionized water to prepare a sodium carbonate solution; and
mixing said silver nitrate solution and said sodium carbonate solution, and stirring for 25 minutes to 35 minutes.

14. The method according to claim 13, wherein said silver ions comprise a silver ion concentration range, which is 10,000 parts per million (ppm) to 100,000 ppm, and titanium ions comprise a titanium ion concentration range, which is 100 ppm to 1,000 ppm.

15. A method for preparing and manufacturing a solution containing a plurality of metal ions, and fluoride ions, comprising:
adding 0.49 ml to 0.59 ml of (3-aminopropyl)triethoxysilane and 0.5 ml to 1.5 ml of aqueous ammonia to 2 ml to 6 ml of deionized water, and stirring for 55 minutes to 65 minutes to form a silicon-containing solution;
performing a centrifugal washing at 8500 to 9500 revolutions per minute for 5 minutes to 15 minutes, perform said centrifugal washing 2 to 5 times, removing a supernatant, and leave a precipitate;
adding 2 ml to 4 ml of hydrogen fluoride or hydrogen chloride and 2 ml to 4 ml of deionized water dropwise to said precipitate to form a precipitate solution; and
adjusting said precipitate solution to a pH of 8 to 12 with ammonia water.

16. The method according to claim 15, wherein said plurality of metal ions comprises silicon ions.

17. The method according to claim 15, wherein said fluoride ion solution further comprises chloride ion solution.

18. The method according to claim 15, when said plurality of metal ions comprises silver ions and silicon ions, wherein, after a step for adding 0.49 ml to 0.59 ml of (3-aminopropyl)triethoxysilane and 0.5 ml to 1.5 ml of aqueous ammonia to 2 ml to 6 ml of deionized water, and stirring for 55 minutes to 65 minutes to form a silicon-containing solution, adding following steps as:
dissolving 1.02 g to 1.12 g of silver nitrate in 1 ml to 5 ml of deionized water to prepare a silver nitrate solution;
dissolving 0.63 g to 0.73 g of sodium carbonate in 5 ml to 15 ml of deionized water to prepare a sodium carbonate solution; and
mixing said silver nitrate solution and said sodium carbonate solution, and stirring for 25 to 35 minutes”.

19. The method according to claim 18, wherein said silver ions comprise a silver ion concentration range, which is 10,000 parts per million (ppm) to 100,000 ppm, and titanium ions comprise a titanium ion concentration range, which is 100 ppm to 1,000 ppm.
